(19) United States
(12) Patent Application Publication
(10) Pub. No.: US 2014/0276687 A1
(43) Pub. Date: Sep. 18, 2014

(54) ASSESSMENT OF VARICOSE VEIN ABLATION VIA IMAGING OR FUNCTIONAL MEASUREMENT ANALYSIS

(71) Applicant: VOLCANO CORPORATION, San Diego, CA (US)

(72) Inventors: David Goodman, Carlsbad, CA (US); Neil Hattangadi, San Diego, CA (US); Gil Laroya, San Diego, CA (US)

(73) Assignee: VOLCANO CORPORATION, San Diego, CA (US)

(21) Appl. No.: 14/202,740

(22) Filed: Mar. 10, 2014

Related U.S. Application Data
(60) Provisional application No. 61/792,407, filed on Mar. 15, 2013.

(51) Int. Cl. A61B 18/24; A61B 18/18

(52) U.S. Cl. CPC A61B 18/24; A61B 18/18
USPC 606/10; 606/33

(57) ABSTRACT

The present invention generally relates to methods for assessing the completeness of a varicose vein ablation. The method involves the use of imaging and/or functional measurement data to determine the extent to which a varicose vein has closed as a result of an ablation procedure.

### RELATED APPLICATION

This application claims the benefit of and priority to U.S. Provisional No. 61/792,407, filed Mar. 15, 2013, which is incorporated by reference in its entirety.

### FIELD OF THE INVENTION

The present invention generally relates to methods for determining the effectiveness of a varicose vein ablation procedure.

### BACKGROUND

Healthy veins contain valves that work against gravity, opening and closing to return blood from the legs back to the heart to be oxygenated. Varicose veins develop when the valves that keep blood flowing in the veins close to the skin become damaged or diseased. Not too long ago, treatment of varicose veins often meant surgically removing or stripping the vein.

A recent advancement in the treatment of varicose veins is varicose vein ablation. In this procedure, a thin catheter is placed into the diseased vein. A stream of energy, generated by either a laser or radiofrequency (RF), is sent through the catheter, causing damage to the internal vessel wall. As the catheter is withdrawn, the vein shrinks and closes. Once the diseased vein is closed, blood is rerouted to other healthy veins.

Despite advancements made in this area, there is still an unmet need to assess the completeness of the ablation. Complications can result from the procedure, including the formation of blood clots in the deep veins of the leg, also known as deep vein thrombosis (DVT). DVT is potentially fatal if the clots break free from the leg and travel to the lungs. Accordingly, it is critical to evaluate whether the diseased vein has been adequately closed and to determine whether any vessel-occluding thrombi have formed as a result of the procedure.

### SUMMARY

The present invention provides a method for assessing the completeness of a varicose vein ablation that uses imaging and/or functional measurement data to assess the completeness of the procedure. The method can involve assessing the vein via imaging or the measurement of functional parameters before and after performing the ablation and comparing the assessments in order to determine the extent to which the vessel has closed after ablation. In other aspects, assessing the vein prior to ablation is not required and the completeness of the procedure can be determined simply by assessing the vein after the procedure has been performed. In additional aspects, the invention can involve assessment of the vein during ablation.

As noted above, the vein may be assessed using intravascular imaging, functional measurements, or any combination of these methods. Intravascular imaging can include, for example, the use of optical coherence tomography (OCT) or intravascular ultrasound (IVUS). In a preferred aspect of the invention, the vein is imaged prior to the ablation using intravascular ultrasound (IVUS). This can involve inserting an IVUS catheter into the vein prior to ablating the vessel and assessing the relative openness of the vessel. The ablation is performed and the IVUS catheter is again inserted into the vessel to determine the extent to which the vessel has closed after the procedure. Any clots resulting from the procedure can also be detected using IVUS. As noted above, the assessment prior to ablation can be omitted when it is clear that the vessel has adequately closed based on the post-procedure image alone. In this instance, the IVUS catheter is inserted into the vessel after ablation has been performed to determine the extent to which the vessel has closed after ablation.

Functional measurements are also suitable for determining whether the vein has closed after ablation. Functional measurements can include, for example, determining the pressure and/or flow inside a vessel before and after ablation, or simply just after ablation. Other suitable functional measurements can include further manipulations of the pressure and/or flow data, including without limitation, fractional flow reserve (FFR), instantaneous wave-free ratio (iFR), coronary flow reserve (CFR), etc. As encompassed by the invention, a drop in pressure and/or flow in the vessel after ablation relative to the initial assessment can indicate vein closure. Where the method involves a single assessment after ablation, the lack of any detectable flow or pressure in the vessel may indicate that the vessel has been successfully closed after ablation. The collection of functional measurements typically involves inserting a pressure, flow, or combination wire into the vessel to take the functional measurement.

Methods of the invention also encompass assessment of the vein during ablation. In this instance, the ablation catheter used to perform the procedure (e.g., an RF or laser ablation catheter), features an imaging sensor and/or a pressure/flow sensor. In this manner, the ablation catheter can be used to both shrink the vessel and collect image/physiological data as the procedure is performed. If data acquisition is performed in real-time along with the ablation, the operator can determine how long to ablate in order to sufficiently desiccate the tissue.

Methods of the invention mitigate the risks associated with varicose vein ablation, resulting in a safer procedure. By monitoring the ablation using image or functional data, the physician can avoid over-ablating and damaging healthy tissue. The physician can also ensure that the vessel has been closed, thereby confirming the success of the procedure. In addition, the physician can determined whether any thrombi have formed as a result of the procedure and take immediate interventional measures.

### BRIEF DESCRIPTION OF THE DRAWINGS

FIG. 1 illustrates a rotational imaging catheter suitable for use in methods of the invention.

FIG. 2 illustrates a phased-array imaging catheter suitable for use in methods of the invention.

FIG. 3 illustrates a distal portion of a detection guidewire suitable for use in methods of the invention.

FIG. 4 illustrates a system suitable for use with detection catheters or guidewires/

FIG. 5 illustrates a distal portion of an ablation catheter according to certain embodiments.

FIG. 6 illustrates a distal portion of another ablation catheter according to certain embodiments.

### DETAILED DESCRIPTION

The present invention provides methods for assessing the completeness of a varicose vein ablation by, for example, determining the extent to which the vessel has closed as a result of the procedure. In certain aspects, the method can involve assessing the vein prior to performing the ablation, performing the ablation, assessing the ablation after performing the ablation, and comparing the pre- and post-assessments, thereby determining the effectiveness of the procedure. In other aspects of the invention, assessing the vein prior to ablation is not required and the completeness of the procedure can be determined simply by assessing the vein after the procedure has been performed. In additional aspects, the invention can involve assessment of the vein during ablation.

In certain aspects, methods of the invention involve assessing the vessel with a detection device. Vessel assessment can be performed both before and after performing the ablation in order to evaluate how much the vessel has closed after ablating. Vessel assessment can also be performed solely after the ablation procedure. This is because the information provided in the post-ablation assessment is sufficient to determine whether the vessel has adequately closed. In certain embodiments, the vessel can be assessed during the ablation procedure.

The detection device is typically an intravascular catheter or guidewire. For example, the detection device can be an imaging catheter or guidewire configured for insertion inside a vessel.

In a typical procedure, the imaging catheter or guidewire is delivered to the tissue of interest via an introducer sheath placed in the radial, brachial or femoral artery. The introducer is inserted into the artery with a large needle, and after the needle is removed, the introducer provides access for guidewires, catheters, and other endovascular tools. An experienced cardiologist can perform a variety of procedures through the introducer by inserting tools such as balloon catheters, stents, or cauterization instruments. When the procedure is complete, the introducer is removed, and the wound can be secured with suture tape. Of course, methods of the invention may also encompass leaving the introducer in so that an ablation catheter may be introduced after the imaging catheter is removed.

In certain aspects, the detection device is an intravascular ultrasound (IVUS) imaging catheter. IVUS uses a catheter with an ultrasound probe attached at the distal end for use inside the patient. The proximal end of the catheter is attached to computerized ultrasound equipment near the operator. To visualize a vessel via IVUS, angiography is used while a technician/physician positions the tip of a guide wire. The physician steers the guide wire from outside the body, through angiography catheters and into the blood vessel branch to be imaged. Additional detail on IVUS imaging can be found in U.S. Pat. No. 5,771,895; U.S. Pub. 2009/0284332; U.S. Pub. 2009/0195514 A1; U.S. Pub. 2007/0232933; and U.S. Pub. 2005/0249391, the contents of each of which are hereby incorporated by reference in their entirety.

In some embodiments, the detection device is a catheter capable of imaging tissues with optical coherence tomography (OCT). OCT uses interferometric measurements to determine radial distances and tissue compositions. Typical intravascular OCT involves introducing the imaging catheter into a patient's target vessel using standard interventional techniques and tools such as a guide wire, guide catheter, and angiography system. Commercially available OCT systems are employed in diverse applications such as art conservation and diagnostic medicine, e.g., ophthalmology. OCT is also used in interventional cardiology, for example, to help diagnose coronary artery disease. OCT systems and methods are described in U.S. Pub. 2011/0152771; U.S. Pub. 2010/0220334; U.S. Pub. 2009/0043191; U.S. Pub. 2008/0291463; and U.S. Pub. 2008/0180683, the contents of each of which are hereby incorporated by reference in their entirety. Further information is also provided in U.S. Pat. No. 7,813,609 and US Patent Publication No, 20090043191, both of which are incorporated herein by reference in their entireties.

In OCT, a light source delivers a beam of light to an imaging device to image target tissue. Within the light source is an optical amplifier and a tunable filter that allows a user to select a wavelength of light to be amplified. Wavelengths commonly used in medical applications include near-infrared light, for example between about 800 nm and about 1700 nm.

Generally, there are two types of OCT systems, common beam path systems and differential beam path systems, that differ from each other based upon the optical layout of the systems. A common beam path system sends all produced light through a single optical fiber to generate a reference signal and a sample signal whereas a differential beam path system splits the produced light such that a portion of the light is directed to the sample and the other portion is directed to a reference surface. Common beam path interferometers are further described for example in U.S. Pat. No. 7,999,938; U.S. Pat. No. 7,995,210; and U.S. Pat. No. 7,787,127, the contents of each of which are incorporated by reference herein in its entirety.

In a differential beam path system, amplified light from a light source is input into an interferometer with a portion of light directed to a sample and the other portion directed to a reference surface. A distal end of an optical fiber is interfaced with a catheter for interrogation of the target tissue during a catheterization procedure. The reflected light from the tissue is recombined with the signal from the reference surface forming interference fringes (measured by a photovoltaic detector) allowing precise depth-resolved imaging of the target tissue on a micron scale. Exemplary differential beam path interferometers are Mach-Zehnder interferometers and Michelson interferometers. Differential beam path interferometers are further described for example in U.S. Pat. No. 7,783,337; U.S. Pat. No. 6,134,003; and U.S. Pat. No. 6,421,164, the contents of each of which are incorporated by reference herein in its entirety.

For embodiments of the invention involving intravascular imaging, the basic methods encompassed by the invention are generally the same whether using IVUS or OCT. In certain aspects, the imaging catheter is guided to the area to be treated and image data is acquired prior to performing the ablation. In a varicose vein, the vessels are swollen and tortuous. Ablation should cause the vessel to shrink, close, or narrow. Therefore, a successful ablation can be determined by comparing a post-ablation image to the pre-ablation image. A decrease in vessel diameter or appearance of vessel closure in the post-ablation image can indicate a complete or successful ablation procedure. In addition, the post-ablation image can be assessed for the appearance of any clots or thrombi.

In another embodiment of the invention, the vessel is imaged only after ablation has been performed. In this case, if the post-ablation image indicates the complete closure of the vessel, the ablation procedure can be considered a success or complete. The post-ablation image can also be assessed for thrombus formation.

In yet another embodiment of the invention, the vessel is imaged during the ablation procedure. In this instance, the ablation device itself has an imaging sensor, such as an IVUS transducer or an OCT optical sensor. In this manner, as the ablation device is ablating the vessel, the operator can see in real-time the effectiveness of the procedure through the acquired images. For example, as the ablation is being performed, the diameter of the vessel will ideally shrink. The physician can begin to remove the ablation catheter as the acquired images indicate that the vessel has completely closed.

Alternatively, functional data can be collected during the ablation procedure with a detection guidewire. In this aspect, a detection guidewire is positioned into the vessel-to-be treated, and an ablation device is guided to the vessel over the detection guidewire. The ablation device can be used to treat the vessel, and the already positioned detection guidewire can be used to obtain functional flow and/or imaging data of the vessel before, during, or after the procedure.

In other aspects of the invention, the detection device detects functional or physiological information associated with the vessel rather than or in addition to image data. Exemplary physiological parameters include pressure or flow (velocity) inside the vessel. The functional measurements collected from the detection device may be processed to determine clinically relevant measurements, such as Fractional Flow reserve measurements, Coronary Flow reserve measurements, instantaneous wave-free ratio (iFR), combined P-V curves, and to display those measurements along with, e.g. pressure and flow readings, in a functional flow image.

Coronary flow reserve is defined as the ratio of maximal coronary flow with hyperemia to normal flow. Coronary flow reserve signifies the ability of the myocardium to increase blood flow in response to maximal exercise. A ratio at or above 2 is considered normal. Abnormal CFR (a ratio below 2) may indicate blood clots, stenosis, arteriovenous fistulas, abnormal constriction of microarteries, and other potential causes of varicose veins. CFR can also be used to determine whether there is an abnormal surplus of flow through the vessel. Coronary flow reserve measures the velocity of the flow. Fractional flow reserve measure pressure differences across a portion of a vessel to determine whether a level of constriction or stenosis of the vessel will impede oxygen delivery to the heart muscle. Specifically, Fractional flow reserve is a ratio of a level of pressure distal to a portion of a vessel under examination to a level of pressure proximal to a portion of a vessel under examination. Often a cut-off point is 0.75 to 0.80 has been used, in which high values indicate a non-significant stenosis, clot or constriction and lower values indicate a significant blood clot and/or stenosis.

P-V loops provide a framework for understanding cardiac mechanics. Such loops can be generated by real time measurement of pressure and volume within the left ventricle. Several physiologically relevant hemodynamic parameters such as stroke volume, cardiac output, ejection fraction, myocardial contractility, etc. can be determined from these loops. To generate a P-V loop for the left ventricle, the LV pressure is plotted against LV volume at multiple time points during a single cardiac cycle. The presence of a stenosis, blood clot, vessel widening, and constriction can alter the curve/shape of P-V loop from a normal P-V loop.

The instantaneous wave-free ratio (iFR) is a vasodilator-free pressure-only measure of the hemodynamic severity of a coronary stenosis comparable to fractional flow reserve (FFR) in diagnostic categorization.

It has been shown that distal pressure and velocity measurements, particularly regarding the pressure drop-velocity relationship such as Fractional Flow reserve (FFR), Coronary flow reserve (CFR), iFR, and combined P-V curves, reveal information about the severity of the vessel damage leading to the varicose vein. For example, in use, the functional flow device may be advanced to a location on the distal side of the varicose vein. The pressure and flow velocity may then be measured at a first flow state. Then, the flow rate may be significantly decreased, for example after the ablation treatment, and the pressure and flow measured in this second flow state. The pressure and flow relationships at these two flow states are then compared to assess the success of the ablation treatment and provide improved insight as for the need for continued intervention/ablation. The ability to take the pressure and flow measurements at the same location and same time with a combined pressure/flow guidewire, improves the accuracy of these pressure-velocity loops and therefore improves the accuracy of the diagnostic information.

Coronary flow reserve, Fractional flow reserve, iFR, and P-V loops may require measurements taken at different locations in the artery. In order to provide measurements for these parameters, systems and methods of the invention may assess pressure and flow at a first location of the data collector against a second location of the data collector within the vasculature. For example, a first location that is distal to a segment of a vessel under examination and a second location that is proximal to that segment of a vessel.

In order to obtain the physiological data described above, the functional measurement device may be equipped with a pressure sensor, a flow sensor, or any combination thereof. Exemplary functional measurement devices suitable for use in practicing the invention include FloWire Doppler Guidewire and the ComboWire XT Guidewire by Volcano Corporation.

In particular embodiments, a pressure sensor can be mounted on the distal portion of a guidewire. In certain embodiments, the pressure sensor is positioned distal to the compressible and bendable coil segment of the guidewire. This allows the pressure sensor to move along with the along coil segment as bended and away from the longitudinal axis. The pressure sensor can be formed of a crystal semiconductor material having a recess therein and forming a diaphragm bordered by a rim. A reinforcing member is bonded to the crystal and reinforces the rim of the crystal and has a cavity therein underlying the diaphragm and exposed to the diaphragm. A resistor having opposite ends is carried by the crystal and has a portion thereof overlying a portion of the diaphragm. Electrical conductor wires can be connected to opposite ends of the resistor and extend within the flexible elongate member to the proximal portion of the flexible elongate member. Additional details of suitable pressure sensors that may be used with devices of the invention are described in U.S. Pat. No. 6,106,476. U.S. Pat. No. 6,106,476 also describes suitable methods for mounting the pressure sensor 104 within a sensor housing.

A flow sensor can be used to measure blood flow velocity within the vessel, which can be used to assess coronary flow reserve (CFR). The flow sensor can be, for example, an ultrasound transducer, a Doppler flow sensor or any other suitable flow sensor, disposed at or in close proximity to the distal tip of the guidewire. The ultrasound transducer may be any suitable transducer, and may be mounted in the distal end using any conventional method, including the manner described in U.S. Pat. Nos. 5,125,137, 6,551,250 and 5,873,835.

A pressure sensor allows one to obtain pressure measurements within a body lumen. A particular benefit of pressure sensors is that pressure sensors allow one to measure of FFR in vessel. FFR is a comparison of the pressure within a vessel at positions prior to the varicose vein, after the varicose vein. The level of FFR determines the condition of the varicose vein, which allows physicians to more accurately identify the appropriate interventional treatment. Another benefit is that a physician can measure the pressure before and after an intraluminal intervention procedure to determine the impact of the procedure.

The acquisition of functional measurements typically involves the insertion of a pressure, flow, or combination catheter/guidewire into a blood vessel and measuring pressure and/or flow inside the vessel with the device. In practice, measuring pressure and/or flow inside the vessel may also involve injecting a local anesthetic into the skin to numb the area of the patient prior to surgery. A puncture is then made with a needle in either the femoral artery of the groin or the radial artery in the wrist before the provided guidewire is inserted into the arterial puncture. Once positioned, the guidewire may then be used to measure pressure and/or flow in the vessel.

Once the device is inside the vessel, the vessel can then be assessed using functional data in much the same way that the vessel is assessed using imaging data. Prior to performing the ablation, the pressure and/or flow guidewire is guided to the area to be treated and functional data is acquired. In a varicose vein, the vessels are swollen and tortuous. Ablation should cause the vessel to shrink, close, or narrow. Therefore, a successful ablation can be evidenced by comparing a post-functional measurement to the pre-functional measurement. A decrease in vessel pressure and/or flow or the absence of any detectable flow or pressure can be used to indicate a complete or successful ablation procedure.

In another embodiment of the invention, functional data is collected from the vessel only after ablation has been performed. In this case, if pressure and/or flow within the vessel is non-existent or substantially non-existent, the ablation procedure can be considered a success or complete.

In yet another embodiment of the invention, functional data is collected during the ablation procedure. In this instance, the ablation device itself may also be a functional measurement device, and can include a pressure and/or flow sensor, as described above. In this manner, as the ablation device is ablating the vessel, the operator can determine in real-time the effectiveness of the procedure through the acquired functional. For example, as the ablation is being performed, the pressure and/or flow within the vessel will decrease. The physician can begin to remove the ablation catheter as the acquired data indicates that the vessel has completely closed.

Alternatively, functional data can be collected during the ablation procedure with a detection guidewire. In this aspect, a detection guidewire is positioned into the vessel-to-be treated, and an ablation device is guided to the vessel over the detection guidewire. The ablation device can be used to treat the vessel, and the already positioned detection guidewire can be used to obtain functional flow and/or imaging data of the vessel before, during, or after the procedure.

Ablation will now be discussed. Methods of the invention encompass performing an ablation after assessing the vessel with a detection device and before re-assessing the vessel with the detection device. Methods of the invention also encompass performing an ablation prior to any assessment of the vessel with a detection device. Methods of the invention further encompass performing an ablation during assessment of the vessel. By taking into account the information provided by the detection device (which in certain aspects, may also be the ablation device), the completeness (effectiveness, success) of the ablation procedure can be determined.

Ablation procedures typically involve contacting a tissue with a hot tool, such as a catheter, or fluid. The heating process often kills the outermost layer of cells contacting the object, and may damage or modify layers of cells below the outermost layer. Some ablation procedures use directed energy to heat and modify the outermost layer of cells, or a nearby layer of cells (treatment depth). In some embodiments, lasers, microwaves, or radiofrequency (RF) waves are directed at the tissue, causing the tissue to heat to treatment temperatures. Typically, the energy is absorbed directly, thus causing the tissue to heat. In some embodiments, a secondary structure, e.g., an antenna, receives the directed energy and heats the tissues. During a procedure the temperature of the tissue is typically elevated to 50° C. or greater, e.g., 55° C. or greater, e.g., 60° C. or greater, e.g., 65° C. or greater, e.g., 70° C. or greater. In some embodiments the tissue is heated to about 65° C., e.g., 68° C.

Venous ablation for varicose veins can be effected in two ways, i.e. percutaneously and endovenously. With the percutaneous approach, the superficial smaller varicose veins and spider veins are “heated” and coagulated by shining an external laser light through the skin. However, if the veins are too large, the amount of energy needed to destroy the veins may result in damage to the surrounding tissues.

With endovenous ablation, a special laser or radio-frequency (RF) catheter is introduced, with local anesthesia, through a needle puncture into the main superficial vein (i.e., the saphenous vein) of the leg. Of course, an entry point may have already been made during insertion of the detection device, as described earlier. Entry is made in the region around the knee, and the catheter is passed up towards the groin, advancing to the site where the saphenous vein joins the deep veins at the site of the main “leaky” valves. Then, as the catheter is slowly withdrawn back through the vein, the laser light or radio-frequency (RF) energy heats up the wall of the vein, endoluminally coagulating the proteins and destroying the lining surface of the vein. The destruction of the lining surface of the vein causes the vein walls to adhere to one another, thereby eliminating the lumen within the vein and thus preventing the flow of blood.

The advantages of endovenous laser/radio-frequency (RF) therapy include: (i) it is a minimally invasive procedure and can be done with local anesthesia, either in an operating room or a physician's office; (ii) it does not require hospitalization; (iii) it does not require open surgery with incisions; (iv) recovery is easier than with open surgery, inasmuch as most patients are back at work within a day or two; and (v) some of the prominent varicosities may disappear and may not require a secondary procedure (i.e., either phlebectomy or sclerotherapy).

The disadvantages of endovenous laser/radio-frequency (RF) therapy include: (i) generally, only one leg is done at a time; (ii) the procedure typically requires significant volumes of local anesthetic to be injected into the patient in order to prevent the complications of the heat necessary to destroy the lining of the vein; (iii) if too much heat is applied to the tissue, there can be burning in the overlying skin, with possible disfiguring, including scarring; (iv) prior to the performance of a subsequent phlebectomy procedure, an interval of up to 8 weeks is required in order to evaluate the effectiveness of the venous ablation procedure; and (v) varicosities that remain after this interval procedure still require separate procedures (i.e., phlebectomy or sclerothapy).

Reference will now be made to an exemplary varicose vein ablation procedure. Further information regarding varicose vein ablation procedures and devices for use in practicing the invention can be found in WO2000/044296; US2012/0283758; US2012/0253192; US2013/0030410; US2011/0238061; U.S. Pat. No. 7,921,854; and U.S. Pat. No. 3,301,258, each of which is incorporated herein by reference.

To begin the procedure, the target vein is accessed using a standard Seldinger technique well known in the art. Under ultrasonic guidance, a small gauge needle is used to puncture the skin and access the vein. A 0.018 inches guidewire is advanced into the vein through the lumen of the needle. The needle is then removed leaving the guidewire in place.

A micropuncture sheath/dilator assembly is then introduced into the vein over the guidewire. A micropuncture sheath dilator set, also referred to as an introducer set, is a commonly used medical kit, for accessing a vessel through a percutaneous puncture. The micropuncture sheath set includes a short sheath with internal dilator, typically 5-10 cm in length. This length is sufficient to provide a pathway through the skin and overlying tissue into the vessel, but not long enough to reach distal treatment sites. Once the vein has been access using the micropuncture sheath/dilator set, the dilator and 0.018 inches guidewire are removed, leaving only the micropuncture introducer sheath in place within the vein. A 0.035 inches guidewire is then introduced through the introducer sheath into the vein. The guidewire (such as a pressure/flow guidewire discussed above) is advanced through the vein until its tip is positioned near the sapheno-femoral junction or other starting location within the vein.

After removing the micropuncture sheath, a treatment catheter set is advanced over the 0.035 inches guidewire until its tip is positioned near the sapheno-femoral junction or other reflux point. The guidewire can be used to take functional flow measurements of the vessel to be treated. Unlike the micropuncture introducer sheath, the treatment catheter is of sufficient length to reach the location within the vessel where the laser treatment will begin, typically the sapheno-femoral junction. Typical treatment catheter lengths are 45 and 65 cm. Once the treatment catheter set is correctly positioned within the vessel, the dilator component and guidewire may be removed or partially withdrawn from the treatment catheter.

Typically, the treatment catheter has one or more ablative element located on its distal end. If so, once positioned, the treatment catheter can proceed with emitting ablative energy to narrow/close the varicose vein. In other aspect, the treatment catheter may define a lumen through an ablative element (such as an optical fiber through which ablative (laser) energy is emitted) can be deployed. In this aspect, the ablation element is then inserted into the treatment sheath lumen and advanced until the ablative element is flush with the distal tip of the treatment sheath. A treatment catheter set as described in U.S. patent application Ser. No. 10/836,084, incorporated herein by reference, may be used to correctly position the ablative element within the vessel. The treatment catheter is retracted a set distance to expose the ablative element, typically 1 to 2 cm. If the treatment catheter has a connector lock as described in U.S. Pat. No. 7,033,347, also incorporated herein by reference, the treatment catheter and ablative element are locked together to maintain the 1 to 2 cm fiber distal end exposure during pullback.

The physician may optionally administer tumescent anesthesia along the length of the vein. Tumescent fluid may be injected into the peri-venous anatomical sheath surrounding the vein and/or is injected into the tissue adjacent to the vein, in an amount sufficient to provide the desired anesthetic effect and to thermally insulate the treated vein from adjacent structures including nerves and skin. Once the vein has been sufficiently anesthetized, laser energy is applied to the interior of the diseased vein segment. A laser generator is turned on and the laser light enters the optical fiber from its proximal end. While the laser light is emitting laser light through the emitting face, the treatment catheter/ablative element is withdrawn through the vessel at a pre-determined rate, typically 2-3 millimeters per second. The laser energy travels along the laser fiber shaft through the energy-emitting face of the fiber and into the vein lumen, where the laser energy is absorbed by the blood present in the vessel and, in turn, is converted to thermal energy to substantially uniformly heat the vein wall along a 360 degree circumference, thus damaging the vein wall tissue, causing cell necrosis and ultimately causing collapse/occlusion of the vessel.

The following describes exemplary detection and ablation devices that may be used in accordance with methods of the invention.

Exemplary imaging catheters that may be used to obtain image data for varicose vein assignment before and after ablative treatment are shown in FIGS. 1 and 2. The imaging catheters shown in FIGS. 1 and 2 are IVUS, but it is understood that OCT imaging catheters and other imaging catheters can also be used. The catheter shown in FIG. 1 is a generalized depiction of a phased array imaging catheter. Phased array imaging catheter 400 is typically around 200 cm in total length and can be used to image a variety of vasculature, such as coronary or carotid arteries and veins. Phased array catheter 400 can be shorter, e.g., between 100 and 200 cm, or longer, e.g., between 200 and 400 cm. When the phased array imaging catheter 400 is used, it is inserted into an artery along a guidewire (not shown) to the desired location (i.e. the location of the varicose vein). Typically a portion of catheter, including a distal tip 410, comprises a guidewire lumen (not shown) that mates with the guidewire, allowing the catheter to be deployed by pushing it along the guidewire to its destination. The catheter, riding along the guidewire, can obtain images of tissue at and surrounding the varicose vein.

An imaging assembly 420 proximal to the distal tip 410, includes a set of transducers that image the tissue with ultrasound energy (e.g., 20-50 MHz range) and a set of image collectors that collect the returned energy (echo) to create an intravascular image. The array is arranged in a cylindrical pattern, allowing the imaging assembly 420 to image 360° inside a vessel. In some embodiment, the transducers producing the energy and the collectors receiving the echoes are the same elements, e.g., piezoelectric elements. Because the phased array imaging catheter 400 does not have a rotating imaging assembly 420, the phased array imaging catheter 400 does not experience non-uniform rotation distortion.

Suitable phased array imaging catheters, which may be used to assess vascular access sites and characterize biological tissue located therein, include Volcano Corporation's Eagle Eye® Platinum Catheter, Eagle Eye® Platinum Short-Tip Catheter, and Eagle Eye® Gold Catheter.

FIG. 2 is a generalized depiction of a rotational imaging catheter 500 incorporating a proximal shaft and a distal shaft of the invention. Rotational imaging catheter 500 is typically around 150 cm in total length and can be used to image a variety of vasculature, such as coronary or carotid arteries and veins. When the rotational imaging catheter 500 is used, it is inserted into an artery along a guidewire (such as a pressure/flow guidewire) to the desired location. Typically a portion of catheter, including a distal tip 510, comprises a lumen (not shown) that mates with the guidewire, allowing the catheter to be deployed by pushing it along the guidewire to its destination.

An imaging assembly 520 proximal to the distal tip 510, includes transducers that image the tissue with ultrasound energy (e.g., 20-50 MHz range) and image collectors that collect the returned energy (echo) to create an intravascular image. The imaging assembly 520 is configured to rotate and travel longitudinally within distal shaft 530 allowing the imaging assembly 520 to obtain 360° images of vasculature over the distance of travel. The imaging assembly is rotated and manipulated longitudinally by a drive cable (not shown). In some embodiments of rotational imaging catheter 500, the distal shaft 530 can be over 15 cm long, and the imaging assembly 520 can rotate and travel most of this distance, providing thousands of images along the travel. Because of this extended length of travel, the speed of the acoustic waves through distal shaft 530 should ideally be properly matched, and that the interior surface of distal shaft 530 has a low coefficient of friction. In order to make locating the distal shaft 530 easier using angioscopy, distal shaft 530 optionally has radiopaque markers 537 spaced apart at 1 cm intervals.

Rotational imaging catheter 500 additionally includes proximal shaft 540 connecting the distal shaft 530 containing the imaging assembly 520 to the ex-corporal portions of the catheter. Proximal shaft 540 may be 100 cm long or longer. The proximal shaft 540 combines longitudinal stiffness with axial flexibility, thereby allowing a user to easily feed the catheter 500 along a guidewire and around tortuous curves and branching within the vasculature. The interior surface of the proximal shaft also has a low coefficient of friction, to reduce NURD, as discussed in greater detail above. The ex-corporal portion of the proximal shaft 540 may include shaft markers that indicate the maximum insertion lengths for the brachial or femoral arteries. The ex-corporal portion of catheter 500 also include a transition shaft 550 coupled to a coupling 560 that defines the external telescope section 565. The external telescope section 565 corresponds to the pullback travel, which is on the order of 150 mm. The end of the telescope section is defined by the connector 570 which allows the catheter 500 to be interfaced to an interface module which includes electrical connections to supply the power to the transducer and to receive images from the image collector. The connector 570 also includes mechanical connections to rotate the imaging assembly 520. When used clinically, pullback of the imaging assembly is also automated with a calibrated pullback device (not shown) which operates between coupling 560 and connector 570.

The imaging assembly 520 produces ultrasound energy and receives echoes from which real time ultrasound images of a thin section of the blood vessel are produced. The transducers in the assembly may be constructed from piezoelectric components that produce sound energy at 20-50 MHz. An image collector may comprise separate piezoelectric elements that receive the ultrasound energy that is reflected from the vasculature. Alternative embodiments of the imaging assembly 520 may use the same piezoelectric components to produce and receive the ultrasonic energy, for example, by using pulsed ultrasound. Another alternative embodiment may incorporate ultrasound absorbing materials and ultrasound lenses to increase signal to noise.

Suitable rotational IVUS catheters, which may be used to assess vascular access sites and characterize biological tissue located therein, include Volcano Corporation's Revolution® 45 MHz Catheter.

The imaging catheters of FIGS. 1 and 2 may also one or more functional sensors. Functional sensors may include pressure sensors, flow sensors, or combinations thereof. In this manner, the imaging catheters may be used to obtain both imaging data and functional data regarding the treatment site before and after treatment. Various functional sensors are discussed in more detail in relation to an exemplary detection guidewire. It is understood that those functional sensors may adapted for use with detection guidewires or catheters.

Exemplary guidewires suitable for use in methods of the invention include guidewires with one or more functional sensors. FIG. 3 shows a sensor tip 700 of a guidewire 401 that may be suitable to use with methods of the invention. Guidewire 401 will include one of pressure sensor 404 and ultrasound transducer 501. In general, guidewire 401 will sensor housing 403 for pressure sensor 404, ultrasound transducer 501, or both and may optionally include a radiopaque tip coil 405 distal to proximal coil 406. The radiopaque tip coil allows one to visualize the guidewire in angiograms.

Pressure sensor 404 can detect a lack of a pressure gradient, indicating that the fistula is not restrictive enough (i.e., if blood flows through the fistula too freely, it will not also flow to distal extremities of that limb of the body, leading to distal ischemia). It may be found, for example, that a ΔP of less than 20 or 30 mmHg is problematic. Pressure sensors and their use are described in U.S. Pub. 2009/0088650 to Corl. Ultrasound transducer 501 may include a forward-looking IVUS and can give the velocity of flow. Velocity data may be derived by the computer in the system from the Doppler frequency shifts detected in the ultrasound echo signals. Obtaining Doppler velocity is discussed in U.S. Pub. 2013/0303907 to Corl and U.S. Pub. 2007/0016034 to Donaldson. While the pressure sensor 404 and ultrasound transducer 501 are described as components of a guidewire, it is contemplated that the pressure sensor and ultrasound can transducer can also be incorporated into an imaging guidewire.

Guidewire 700 may comprise a flexible elongate element having proximal and distal ends and a diameter of 0.018″ or less as disclosed in U.S. Pat. No. 5,125,137, U.S. Pat. No. 5,163,445, U.S. Pat. No. 5,174,295, U.S. Pat. No. 5,178,159, U.S. Pat. No. 5,226,421, U.S. Pat. No. 5,240,437 and U.S. Pat. No. 6,106,476, all of which are incorporated by reference herein. Guidewire 700 can be formed of a suitable material such as stainless steel, Nitinol, polyimide, PEEK or other metallic or polymeric materials having an outside diameter for example of 0.018″ or less and having a suitable wall thickness, such as, e.g., 0.001″ to 0.002″. This flexible elongate element is conventionally called a hypotube. In one embodiment, the hypotube may have a length of 130 to 170 cm. Typically, such a guide wire may further include a stainless steel core wire extending from the proximal extremity to the distal extremity of the flexible elongate element to provide the desired torsional properties to facilitate steering of the guide wire in the vessel and to provide strength to the guidewire and prevent kinking.

In a preferred embodiment, methods of the invention employ a Doppler guidewire wire sold under the name FLOWIRE by Volcano Corporation, the pressure guidewire sold under the name PRIMEWIRE PRESTIGE by Volcano Corporation, or both.

Referring now to FIG. 4, the detection catheter 400, 500 or guidewire 700 may be coupled to and coordinated by a system controller 600. The system controller 600 may control the timing, duration, and amount of imaging and functional data collection. As shown in FIG. 4, the system controller 600 is additionally interfaced with processing computer 1060 that processes the obtained image and functional flow data. In certain embodiments, the system controller is associated with separate processing computers for imaging and functional flow data processing. According to certain embodiments, the processor 1065 of the processing computer 1060 performs tissue/blood characterization, thereby allowing the viewed and assessed images to be the basis for defining parameters used to develop a therapeutic mode for treating the varicose vein. The system 1000 also includes a display 580 and a user interface that allow a user, e.g. a surgeon, to interact with the images/functional data and to control the parameters of the treatment.

As shown in FIG. 4, the system controller 600 is interfaced to a processing computer 1060 that is capable of synthesizing the images and tissue measurements into easy-to-understand images. The processing computer 1060 also provides functional flow readouts for display onto a screen. The processing computer is also configured to analyze the spectrum of the collected data to determine tissue characteristics, a.k.a. virtual histology. As discussed in greater detail below, the image processing will deconvolve the reflected acoustic waves or interfered infrared waves to produce distance and/or tissue measurements, and those distance and tissue measurements can be used to produce an image, for example an IVUS image or an OCT image. Flow detection and tissue characterization algorithms, including motion-detection algorithms (such as CHROMAFLO (IVUS fluid flow display software; Volcano Corporation), Q-Flow, B-Flow, Delta-Phase, Doppler, Power Doppler, etc.), temporal algorithms, harmonic signal processing, can be used to differentiate blood speckle from other structural tissue, and therefore enhance images where ultrasound energy back scattered from blood causes image artifacts.

In certain aspects, methods of the invention involve an ablation catheter used to treat the varicose vein. In some instances, the ablation tool can be extended from the catheter lumen and into a vessel, such as a blood vessel, to perform ablation therapy. In certain embodiments, the ablation catheter may also include one or more imaging sensors or functional sensors. In this manner, the ablation catheter may provide imaging to allow the field of therapy to be observed before, during, and after the ablation therapy. For example, a therapy catheter can be configured to image the ablation procedure performed along the side of the catheter while imaging the treatment tissue with a proximal (or distal) imaging element.

There are several different types of ablation therapies. In one aspect, an ablation tool is used to remove an unwanted or damaged vein by delivering energy (RF energy, laser energy, etc.) within a vein to shrink and ultimately close the vein. In some instances, the proximal end of the ablation tool is connected to an energy source that provides energy to the electrodes for ablation. The energy necessary to ablate cardiac tissue and create a permanent lesion can be provided from a number of different sources including radiofrequency, laser, microwave, ultrasound and forms of direct current (high energy, low energy and fulgutronization procedures). Any source of energy is suitable for use in the ablation tool of the invention. In some embodiments, the ablation tool includes at least one electrode. The electrodes can be arranged in many different patterns along the ablation tool. For example, the electrode may be located on a distal end of the ablation tool. In addition, the electrodes may have a variety of different shape and sizes. For example, the electrode can be a conductive plate, a conductive ring, conductive loop, or a conductive coil. In one embodiment, the at least one electrode includes a plurality of wire electrodes configured to extend out of the distal end of the imaging electrode.

FIGS. 5-6 depict the distal end of ablation catheters suitable for use with the methods of the invention. As depicted in FIG. 5, the ablation catheter 200 includes a catheter body 202 that defines a lumen through which an ablation element 204 can be extended. The ablation element 204 is deployable from an opening 206 at the distal end of the catheter body 204. The ablation element 204 may be, for example, an optical fiber configured to emit laser energy. Alternatively, the ablation element may be an electrode, such as metal plating, configured to emit radio-frequency. FIG. 6 illustrates an ablation catheter 300 that includes an electrode as the ablation element. As shown in FIG. 6, the distal portion of the ablation catheter 300 includes an electrode 304 positioned on the surface of the catheter body 302. While shown in a spiral configuration, the electrode 304 may be positioned on the catheter body 302 in any suitable manner. The electrode 304 may be configured to deliver radiofrequency energy or any other energy suitable for treating varicose veins. As discussed above, the ablation catheters of FIGS. 5 and 6 can also include imaging or functional sensors, which allows for data collection during the ablation procedure.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method for assessing the completeness of an ablation procedure, the method comprising:
inserting a detection device inside a vessel and acquiring a first set of detection data using the device;
inserting an ablation device into the vessel;
performing an ablation with said ablation device;
re-inserting the detection device inside the vessel and acquiring a second set of detection data using the device; and
comparing the second set of detection data to the first set, thereby assessing the completeness of the ablation procedure.

2. The method of claim 1, wherein the detection device comprises an imaging catheter or guidewire and the detection data comprises image data.

3. The method of claim 2, wherein imaging comprises intravascular ultrasound (IVUS) imaging or optical coherence tomography (OCT) imaging.

4. The method of claim 1, wherein the detection device comprises a pressure-sensing guidewire, a flow-sensing guidewire, or a combination pressure/flow-sensing wire and the detection data comprises functional measurement data.

5. The method of claim 4, wherein the functional measurement comprises pressure, flow, fractional flow reserve (FFR), coronary flow reserve (CFR), or instantaneous wave-free radio (iFR).

6. The method of claim 1, wherein ablation comprises laser ablation or radiofrequency ablation.

7. The method of claim 1, wherein the ablation device is an ablation catheter.

8. The method of claim 1, wherein the vessel is a varicose vein.

9. The method of claim 1, wherein assessing the completeness of the ablation procedure comprises determining the extent to which the vessel has closed subsequent to the ablation procedure.

10. A method for assessing the completeness of an ablation procedure, the method comprising:
inserting an ablation device into a vessel;
performing an ablation with said ablation device;
inserting a detection device inside the vessel and acquiring a set of detection data using the device; and
assessing the completeness of the ablation procedure based on the acquired set of detection data.

11. The method of claim 10, wherein the ablation device is an ablation catheter.

12. The method of claim 11, wherein ablation comprises laser ablation or radiofrequency ablation.

13. The method of claim 10, wherein the detection device comprises an imaging catheter or guidewire and the detection data comprises image data.

14. The method of claim 13, wherein imaging comprises intravascular ultrasound (IVUS) imaging or optical coherence tomography (OCT) imaging.

15. The method of claim 10, wherein the detection device comprises a pressure-sensing guidewire, a flow-sensing guidewire, or a combination pressure/flow-sensing wire and the detection data comprises functional measurement data.

16. The method of claim 15, wherein the functional measurement comprises pressure, flow, fractional flow reserve (FFR), coronary flow reserve (CFR), or instantaneous wave-free radio (iFR).

17. The method of claim 10, wherein the vessel is a varicose vein.

18. The method of claim 10, wherein assessing the completeness of the ablation procedure comprises determining the extent to which the vessel has closed subsequent to the ablation procedure.

19. A method for assessing the completeness of an ablation procedure, the method comprising:
inserting an ablation device into a vessel, wherein said ablation device has an imaging sensor or functional measurement sensor positioned thereon;
imaging the vessel or acquiring a functional measurement within the vessel with the ablation device; and
while imaging or acquiring said functional measurement;
performing the ablation; thereby assessing the completeness of the ablation procedure in real-time based on said imaging or acquisition of functional measurement.

20. The method of claim 19, wherein the ablation device is an ablation catheter.

21. The method of claim 19, wherein ablation comprises laser ablation or radiofrequency ablation.

22. The method of claim 19, wherein said imaging comprises intravascular ultrasound (IVUS) imaging or optical coherence tomography (OCT) imaging.

23. The method of claim 15, wherein the functional measurement comprises pressure, flow, fractional flow reserve (FFR), coronary flow reserve (CFR), or instantaneous wave-free radio (iFR).

24. The method of claim 19, wherein the vessel is a varicose vein.

25. The method of claim 19, wherein assessing the completion of the ablation procedure comprises determining the extent to which the vessel has closed as a result of the ablation procedure.
